(19) United States
(12) Patent Application Publication
(10) Pub. No.: US 2010/0151021 A1
(43) Pub. Date: Jun. 17, 2010

(54) Compositions Comprising Melperone

(76) Inventors: Gopi M. Venkatesh, Vandalia, OH (US); Phillip J. Stevens, Englewood, OH (US); Jin-Wang Lai, Springborro, OH (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/639,496

(22) Filed: Dec. 16, 2009

Related U.S. Application Data
(60) Provisional application No. 61/122,824, filed on Dec. 16, 2008.

(51) Int. Cl. A61K 31/4515; A61K 9/30; A61K 9/14; A61K 9/22; A61P 25/18; A61P 25/16

(52) U.S. Cl. 424/465; 424/490; 424/493; 424/494; 424/495; 424/497; 514/317

(57) ABSTRACT

The present invention is directed to pharmaceutical compositions, and methods of making such compositions, comprising microparticles containing melperone and/or a pharmaceutically acceptable salt, solvate, or ester thereof; a layer of alkaline buffer, and a controlled-release coating. The present invention is also directed to pharmaceutical dosage forms, including orally disintegrating tablets, conventional tablets, and capsules, and methods for their preparation.

### CROSS REFERENCE TO RELATED APPLICATIONS

The present application claims priority to U.S. Provisional Application No. 61/122,824 filed Dec. 16, 2008, which is incorporated herein by reference in its entirety for all purposes.

### BACKGROUND OF THE INVENTION

Parkinson's disease (PD) is a progressive neurodegenerative disorder characterized by bradykinesia, rigidity, tremor, and abnormal posture and gait. The primary pathology of PD is the marked loss (80-90%) of dopaminergic neurons that provide dopaminergic innervation to the striatum, resulting in a potential decrease in dopamine activity and an imbalance in the dopamine/acetylcholine systems that control movement. Long-term treatments of PD movement disorders with dopamine precursors (anti-Parkinsonian agents), although successful, are thought to contribute to psychiatric side effects (psychosis). Psychotic symptoms characteristically observed in PD include hallucinations. Patients with PD who develop psychosis have a higher rate of morbidity and mortality (approaching 100%) and may cause considerable caregiver stress and/or may end up in nursing homes (see Barbato L et al., “Melperone in the treatment of iatrogenic psychosis in Parkinson's disease,” Funct. Neurol. (1996) 11:201-7 and Fernandez H. et al., “Treatment of psychosis in Parkinson's disease: safety considerations,” Drug Saf. (2003); 26(9):643-59, each of which is incorporated in their entirety by reference for all purposes).

Currently there are no compounds approved for the treatment of psychosis associated with PD. Although the use of quetiapine, an atypical antipsychotic agent, has shown efficacy in initial open-label studies, worsening of motor symptoms of PD was observed in up to 30% of patients. More options for treatment of psychosis in patients with PD are needed, particularly treatments that do not contribute to the worsening of motor symptoms in patients who already suffer from poor motor functioning.

In a proof-of-concept study, melperone has shown efficacy in the treatment of psychosis in patients with PD. The onset of efficacy was rapid (within a week), and efficacy with a mean daily dose of 37.5 mg was maintained over 2 years of treatment. Moreover, the motor symptoms were not worsened by melperone treatment. Its greater affinity for serotonin 5-HT2 receptors relative to dopamine D2 receptors, minimal striatal activity, lack of significant clinical effect on plasma prolactin levels, low incidence of extrapyramidal side effect (EPS) or tardive dyskinesia make melperone a particularly attractive candidate to treat psychosis.

Melperone hydrochloride, 1-(4-fluorophenyl)-4-(4-methyl-1-piperidinyl)-1-butanone hydrochloride is a white crystalline powder having a wide spectrum of neuroleptic properties. Melperone hydrochloride has a pKa of 9.1 and a short plasma elimination half life of about 3 hrs upon a single oral dose administration. Its molecular weight is 299.82. Melperone is freely soluble in aqueous buffers at acidic to neutral pH conditions, but is poorly soluble at higher pH values (i.e., above pH=7.5; see the table below for details).

Solubility (mg/mL) of Melperone HCl

| Media | Solubility (mg/mL) |
|---|---|
| pH 1.2 (0.1N HCl) | 700 |
| pH 4.5 (phosphate) | 800 |
| pH 6.8 (phosphate) | 800 |
| Purified Water | 800 |
| pH 7.0 (phosphate) | >100 |
| pH 7.1 (phosphate) | 51.3 |
| pH 7.8 (phosphate) | 25.6 |
| Ethanol | 88.5 |

Oral doses of melperone are rapidly absorbed (mean Tₘₐₓ: approximately 1.5-3.0 hours upon oral tablet administration). Within the 25-100 mg oral dose range, the pharmacokinetic profile is non-linear at higher doses, giving relatively higher Cₘₐₓ and AUC values than would be expected with linear pharmacokinetics. Due to extensive first-pass metabolism, the absolute oral bioavailability is about 50-70%. In healthy subjects, melperone metabolism is also rapid (t_{1/2}=3-4 hrs following single oral doses) and extensive, with only about 7% of the dose excreted unchanged in urine. The t_{1/2} is longer following chronic dosing; t_{1/2} at steady-state was approximately 7 hrs in patients with schizophrenia receiving 100 mg melperone three times daily (tid) and a variety of concomitant medications. Formulations of melperone permitting a once- or twice-daily dosage regimen are desirable in order to improve compliance in patients suffering from schizophrenia or in the treatment of psychosis associated with PD. An orally disintegrating tablet (ODT) formulation containing controlled release melperone particles is especially useful to geriatric patients (who often have difficulty swallowing conventional tablets and capsules) and caregivers of mentally ill patients (who often resist or “cheek” their medications). Controlled release ODT formulations rapidly disintegrate in the oral cavity, and they are easily swallowed without water. Such formulations would reduce the frequency of dosing and ease administration problems.

However, developing oral dosage forms for once-daily administration of weakly basic drugs like melperone is challenging. For example, weakly basic drugs such as melperone dissolve rapidly under acidic conditions and most (if not essentially all) of the drug is released before exiting the stomach. However, once-daily compositions must release the drug throughout the gastrointestinal tract wherein the drug is extremely soluble, thereby requiring relatively thick extended release coatings to reduce the drug release rate. Providing such thick coatings can be problematic, for example they can be difficult to manufacture, and/or increase the bulk of the dosage form (particularly for drugs such as melperone, which require the administration of relatively high doses). Thick extended release coatings can be a particular problem for dosage forms such as orally disintegrating tablets, which require small drug-containing particles (<500 μm average particle size) in order to provide suitable organoleptic properties (e.g., smooth, non-gritty mouthfeel).

### SUMMARY OF THE INVENTION

In one embodiment, the present invention relates to a pharmaceutical composition comprising at least one population of particles, wherein each particle of at least one population of particles comprises a core comprising melperone or a pharmaceutically acceptable salt, ester, and/or solvate thereof; an alkaline buffer layer disposed over the core; and a controlled-release coating disposed over the alkaline buffer layer, wherein the controlled-release coating comprises a water-insoluble polymer.

In another embodiment, the present invention relates to a method of preparing the pharmaceutical composition, comprising (a) preparing a core comprising melperone or a pharmaceutically acceptable salt, ester, and/or solvate thereof; (b) coating the core of step (a) with a layer comprising an alkaline buffer; and (c) coating the alkaline-buffer layered core of step (b) with a controlled-release layer.

In another embodiment, the present invention relates to a pharmaceutical dosage form comprising a plurality of particles. Each particle comprises a core comprising melperone (or a pharmaceutically acceptable salt, ester, and/or solvate thereof); an alkaline buffer layer disposed over the core; and a controlled-release coating disposed over the alkaline buffer layer, wherein the controlled-release coating comprises a water-insoluble polymer, optionally in combination with a water-soluble polymer and/or an enteric polymer.

In yet another embodiment, the present invention relates to a pharmaceutical dosage form comprising at least two populations of drug particles. One population of drug particles comprises melperone-containing particles, while the second population comprises melperone-containing particles, an alkaline buffer layer disposed over the melperone-containing particle, and a controlled-release layer disposed over the alkaline buffer layer. The controlled-release layer comprises a water-insoluble polymer, optionally in combination with an enteric polymer.

In another embodiment, the present invention relates to a pharmaceutical dosage form comprising at least one population of particles. Each particle of at least one population of particles comprises a core comprising melperone and/or a pharmaceutically acceptable salt, ester, or solvate thereof; an alkaline buffer layer disposed over the core; and a controlled-release coating disposed over the alkaline buffer layer, wherein the controlled-release coating comprises a water-insoluble polymer.

In another embodiment, the present invention relates to a method of preparing the pharmaceutical dosage form. In a particular embodiment, the pharmaceutical dosage form is prepared by mixing the melperone-containing particles described herein with rapidly dispersing granules comprising a saccharide and/or a sugar alcohol in combination with a disintegrant to form a compressible blend, and compressing the blend into a tablet. In another embodiment, the pharmaceutical dosage form is prepared by filling the microparticles described herein into a hard-gelatin capsule.

In another embodiment, the present invention relates to a method of preparing a pharmaceutical dosage form comprising at least one population of particles. Each particle of the at least one population of particles comprises a core comprising melperone or a pharmaceutically acceptable salt, ester, and/or solvate thereof; an alkaline buffer layer disposed over the melperone-containing core; and two controlled-release coatings disposed over the alkaline buffer layer, wherein the inner controlled-release coating comprises a water-insoluble polymer and the outer controlled-release coating comprises a water-insoluble polymer in combination with an enteric polymer.

In yet another embodiment, the pharmaceutical dosage form is prepared by mixing the melperone-containing particles described herein with rapidly dispersing granules comprising a saccharide and/or a sugar alcohol in combination with a disintegrant to form a compressible blend, and compressing the blend into an orally disintegrating tablet. In another embodiment, the pharmaceutical dosage form is prepared by filling the microparticles described herein into a hard-gelatin capsule or by blending the microparticles described herein with other pharmaceutically acceptable excipients and compressing into a conventional tablet.

### BRIEF DESCRIPTION OF THE DRAWINGS

FIG. 1 illustrates the cross-section of a SR or a TPR bead comprising an alkaline buffer-coated IR bead comprising melperone or a pharmaceutically acceptable salt, solvate, and/or ester thereof in accordance with a particular aspect of the invention. FIG. 1 (top schematic) depicts an embodiment of an alkaline buffer coated IR bead 10 comprising an alkaline buffer layer 12 disposed over a protective sealant layer 14, which is disposed over a melperone layer 16 disposed over an inert core 18. FIG. 1 (lower schematic) depicts an embodiment of a SR or TPR bead 20 comprising a compressible coating 26 disposed over a SR or TPR coating 24, which is disposed over a sealant layer 22, which is disposed over an alkaline buffer coated IR bead 10 (the IR bead 10 can comprise, e.g., the IR bead of the top schematic of FIG. 1).

FIG. 2 is a schematic of a two-compartment pharmacokinetic model.

FIG. 3 illustrates the deconvoluted in vitro release profiles of melperone hydrochloride of Example 1.

FIG. 4 illustrates the melperone release profiles of SR beads of Example 2.

FIG. 5 illustrates the melperone release profiles of SR beads of Example 3.

FIG. 6 illustrates the melperone release profiles of SR beads and ODT compositions comprising SR beads of Example 3 and ODT CR (PF416EA0001) of Example 4 (and comparative theoretical profiles: slow release at rate constant 0.14 h⁻¹ and fast release at rate constant 0.29 h⁻¹).

FIG. 7 illustrates the melperone release profiles of SR beads and ODT prototypes of Example 6.

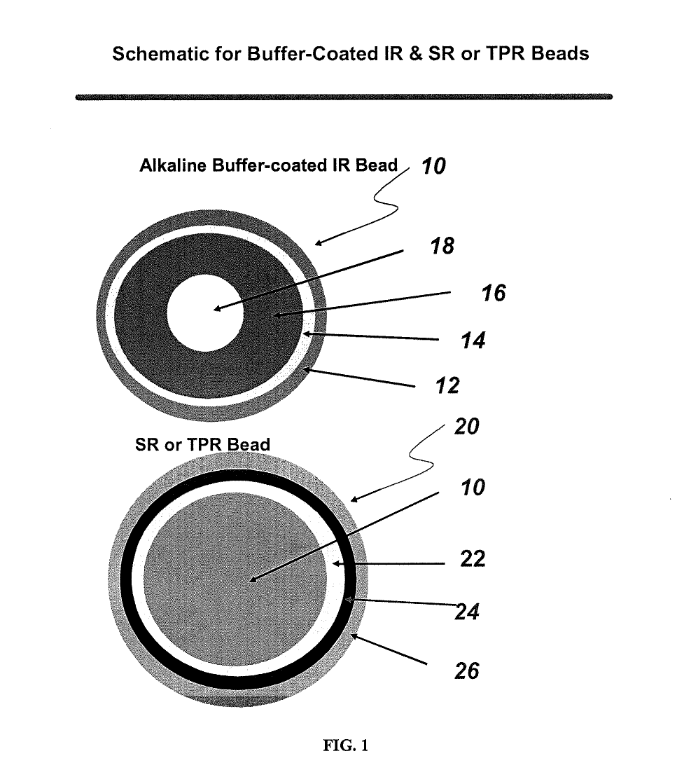

### DETAILED DESCRIPTION OF THE INVENTION

All documents cited are incorporated herein by reference in their entirety for all purposes. The citation of any document is not to be construed as an admission that it is prior art with respect to the present invention.

The term “drug”, “active”, or “active pharmaceutical ingredient” as used herein includes a pharmaceutically acceptable and therapeutically effective compound, pharmaceutically acceptable salts, stereoisomers and mixtures of stereoisomers, solvates (including hydrates), polymorphs, and/or esters thereof. When referring to a drug in the descriptions of the various embodiments of the invention, the reference encompasses the base drug, pharmaceutically acceptable salts, stereoisomers and mixtures of stereoisomers, solvates (including hydrates), polymorphs, and/or esters thereof.

The terms “orally disintegrating tablet” or “ODT” refers to a tablet which disintegrates rapidly in the oral cavity of a patient after administration, without the need for chewing. The rate of disintegration can vary, but is faster than the rate of disintegration of conventional solid dosage forms (e.g., tablets or capsules) which are intended to be swallowed immediately after administration, or chewable solid dosage forms.

The term “about”, as used herein to refer to a numerical quantity, includes “exactly”. For example, “about 60 seconds” includes 60 seconds, exactly, as well as values close to 60 seconds (e.g., 50 seconds, 55 seconds, 59 seconds, 61 seconds, 65 seconds, 70 seconds, etc.).

As used herein, the term “controlled-release” coating encompasses coatings that delay release, sustain release, prevent release, and/or otherwise prolong the release of a drug from a particle coated with a controlled-release coating. The term “controlled-release” encompasses “sustained-release” and “timed, pulsatile release.” As used herein, the term “controlled-release coating” encompasses a timed, pulsatile release or “lag-time” coating.

As used herein, the term “immediate-release core” refers to a core containing a drug, optionally layered with a sealant layer, but not coated with a controlled-release coating. An “immediate-release core” can include drug crystals (or amorphous particles), granules of the drug with one or more excipients, or an inert core (e.g., a sugar sphere) layered with a drug (and an optional binder), a protective sealant coating, and an optional alkaline buffer layer. “Immediate release cores” have immediate release properties as described herein. Extended release particles (e.g., SR particles, TPR particles, etc.) can be prepared by coating immediate-release cores with an extended release coating.

As used herein, the term “immediate release” or IR refers to release of greater than or equal to about 50% (especially if taste-masked for incorporation into an orally disintegrating tablet dosage form), preferably greater than about 75%, more preferably greater than about 90%, and in accordance with certain embodiments greater than about 95% of the active within about 2 hours, more particularly within about one hour following administration of the dosage form.

The term “TPR particle” or “TPR bead” refers to a drug-containing particle, e.g., a drug-layered bead, drug-containing granulate, or drug particle, coated with a TPR (“timed pulsatile release”) coating. The TPR coating provides an immediate release pulse of the drug, or a sustained drug-release profile after a pre-determined lag time. The term “lag-time” refers to a time period immediately after administration of the drug-containing particle wherein less than about 10%, more particularly substantially none, of the drug is released from a particle. In some embodiments, a lag-time of from at least about 2 to 10 hours is achieved by coating the particle with, e.g. a combination of at least one water-insoluble polymer and at least one enteric polymer (e.g., a combination of ethylcellulose and hypromellose phthalate). The TPR layer can optionally contain a plasticizer.

The term “sustained-release coating” or “SR coating” refers to a coating providing sustained-release properties, e.g. a coating which slows the release of the drug from the drug-containing particle but does not provide an appreciable “lag-time.”

The term “substantially disintegrates” means a level of disintegration amounting to disintegration of at least about 50%, at least about 60%, at least about 70%, at least about 80%, at least about 90%, or about 100% disintegration of the ODT composition.

The term “substantially masks the taste” in reference to the taste-masking layer of the IR particles (when present) refers to the ability of the taste-masking layer to substantially prevent release of a bitter tasting drug in the oral cavity of a patient. A taste-masking layer which “substantially masks” the taste of the drug typically releases less than about 10% of the drug in the oral cavity of the patient, in other embodiments, less than about 5%, less than about 1%, less than about 0.5%, less than about 0.1%, less than about 0.05%, less than about 0.03%, less than about 0.01% of the drug. The taste-masking properties of the taste-masking layer of the compositions of the present invention can be measured in vivo (e.g., using conventional organoleptic testing methods known in the art) or in vitro (e.g., using dissolution tests as described herein). The skilled artisan will recognize that the amount of drug release associated with a taste-masking layer that “substantially masks” the taste of a drug is not limited to the ranges expressly disclosed herein, and can vary depending on other factors, such as the perceived the bitterness of the drug and the presence of other flavoring agents in the composition.

The term “rapidly dispersing tablet” as disclosed in this application refers to a conventional tablet that is orally administered either by swallowing or dispersing in a small amount of water. “Rapidly dispersing tablets” comprise at least one drug, and optionally a disintegrant, one or more fillers/diluents (e.g. including microcrystalline cellulose or mannitol). “Rapidly dispersing tablets” are distinguished from ODTs in that ODTs contain the rapidly dispersing microgranules described herein (comprising a saccharide and/or a sugar alcohol in combination with a disintegrant), whereas “rapidly dispersing tablets” do not (even though they may contain disintegrants and sugar alcohols). Because ODTs contain rapidly dispersing microgranules, ODTs disintegrate rapidly in the oral cavity upon exposure to saliva.

The terms “plasma concentration-time profile”, “Cₘₐₓ”, “AUC”, “Tₘₐₓ”, and “elimination half life” have their generally accepted meanings as defined in the FDA Guidance to Industry: Bioequivalence.

Unless indicated otherwise, all percentages and ratios are calculated by weight based on the total composition.

The term “disposed over” means that a second material is deposited over a first material, wherein the second material may or may not be in physical contact with the first material. Thus it is possible, but not necessary, that an intervening material lies between the first and second materials.

In one embodiment, the present invention relates to a pharmaceutical composition comprising a plurality of controlled-release particles, wherein each particle comprises a core comprising melperone and/or a pharmaceutically acceptable salt, solvate, or ester thereof; an alkaline buffer layer disposed over the core; and a controlled-release coating disposed over the alkaline buffer layer. In particular embodiments, the controlled-release coating comprises a water-insoluble polymer. In other embodiments, the controlled-release coating comprises a water-insoluble polymer in combination with a water-soluble polymer. In still other embodiments, the controlled-release coating comprises a water-insoluble polymer in combination with an enteric polymer.

The alkaline buffer layer is believed to create an alkaline microenvironment at the drug interface inside the controlled-release particle. Because the weakly basic drug has a lower solubility in this microenvironment, the alkaline buffer layer effectively delays release of the drug under the acidic to neutral pH conditions of the gastrointestinal tract, conditions under which the drug would otherwise dissolve rapidly. By incorporating an alkaline buffer layer into the compositions of the present invention, it is possible to extend the release of melperone, thereby providing pharmacokinetic profiles suitable for a once-daily dosing regimen for melperone.

Non-limiting examples of alkaline buffers suitable for the compositions of the present invention include sodium hydroxide, monosodium dihydrogen phosphate, disodium hydrogen phosphate, trisodium phosphate, sodium acetate, sodium carbonate or bicarbonate, monopotassium dihydrogen phosphate, dipotassium hydrogen phosphate, tripotassium phosphate, potassium acetate, potassium carbonate or bicarbonate, magnesium phosphate, magnesium acetate, magnesium carbonate, magnesium oxide, magnesium hydroxide, sodium silicate, calcium silicate, complex magnesium aluminum metasilicate, and mixtures thereof.

The alkaline buffer layer optionally contains a polymeric binder. Any pharmaceutically acceptable polymeric binder which is compatible with the alkaline buffer composition may be used. Suitable polymeric binders include for example, polymers selected from the group consisting of hydroxypropylcellulose, povidone, methylcellulose, hydroxypropyl methylcellulose, carboxyalkylcellulose, polyethylene oxide, and a polysaccharide.

Within the controlled-release particle, the alkaline buffer layer can be disposed on one or more layers disposed between the alkaline buffer layer and the melperone. In a particular embodiment, the alkaline buffer layer is disposed on a sealant layer, which in turn is disposed on the melperone-containing core. In one embodiment, the melperone-containing core is a granulate containing melperone. In another embodiment, the melperone-containing core is melperone crystals. In another embodiment, the melperone-containing core is an inert bead (e.g. a sugar sphere) coated with a melperone layer. In some embodiments, the ratio of the alkaline buffer to melperone ranges from about 5:1 to about 1:5, including from about 3:1 to about 1:3.

In certain embodiments, the compositions of the present invention comprise a sealant layer disposed on the melperone-containing core, underlying the alkaline buffer layer. This protective sealant layer separates the melperone-containing core and the alkaline buffer layer and may provide one or more of the following advantages: prevent (or minimize) contact between the melperone and alkaline buffer during processing or storage; prevent (or minimize) static; prevent (or minimize) particle attrition; avoid potential instability issues that may result from the proximity of the melperone and the alkaline buffer during melperone layering or storage (e.g., formation of an addition compound between melperone and buffer); and insure that the alkaline buffer and the melperone do not come into direct contact until the dosage form comes into contact with a dissolution medium or body fluid following oral ingestion. In one embodiment, the sealant layer comprises a hydrophilic polymer. Non-limiting examples of suitable hydrophilic polymers include hydrophilic hydroxypropylcellulose (e.g., Klucel® LF), hydroxypropyl methylcellulose or hypromellose (e.g., Opadry® Clear or Pharmacoat™ 603), vinylpyrrolidone-vinylacetate copolymer (e.g., Kollidon® VA 64 from BASF), and ethylcellulose, e.g. low-viscosity ethylcellulose. The sealant layer can constitute from about 1% to about 10% of the weight of the melperone-containing, sealant-coated core, for example about 1%, about 2%, about 3%, about 4%, about 5%, about 7%, about 8%, about 8%, or about 10%, inclusive of all ranges and subranges therebetween.

In some embodiments, the microparticles of the present invention comprise a controlled-release coating disposed on the alkaline buffer layer, comprising a water-insoluble polymer. In one embodiment, the controlled-release coating comprises the water-insoluble polymer in the absence of a water-soluble or enteric polymer. In this embodiment, the controlled-release coating sustains release of melperone over about 8 hours to about 20 hours, when tested in the two-stage dissolution method (700 mL of 0.1N HCl (hydrochloric acid) for the first 2 hours and thereafter in 900 mL at pH 6.8 obtained by adding 200 mL of a pH modifier). This release profile is suitable for a once- or twice-daily dosing regimen.

In another embodiment, the controlled release coating comprises a water-insoluble polymer in combination with a gastrosoluble pore-former. An example of a gastrosoluble pore-former is calcium carbonate. Other suitable gastrosoluble pore-formers include sodium chloride, calcium phosphate, calcium saccharide, calcium succinate, calcium tartrate, ferric acetate, ferric hydroxide, ferric phosphate, magnesium carbonate, magnesium citrate, magnesium hydroxide, magnesium phosphate, etc.

Non-limiting examples of suitable water-insoluble polymers include ethylcellulose, cellulose acetate, cellulose acetate butyrate, polyvinyl acetate, neutral methacrylic acid-methylmethacrylate copolymers, and mixtures thereof. In one embodiment, the water-insoluble polymer comprises ethylcellulose. In another embodiment, the water-insoluble polymer comprises ethylcellulose with a mean viscosity of 10 cps in a 5% solution in 80/20 toluene/alcohol measured at 25° C. on an Ubbelohde viscometer. The water-insoluble polymer of the sustained-release coating provides a weight gain from about 3% to about 40%, including about 3%, about 5%, about 7%, about 10%, about 12%, about 15%, about 17%, about 20%, about 22%, about 25%, about 27%, about 30%, about 35%, and about 40%, inclusive of all ranges and subranges therebetween. In one example, the sustained-release microparticle may have a sustained-release coating of a plasticized water-insoluble polymer, such as ethylcellulose (EC-10), at about 5-50% by weight to sustain the release of melperone over about 4-20 hours.

In one embodiment, the water-insoluble polymer of the controlled-release coating further comprises a plasticizer. Non-limiting examples of suitable plasticizers include triacetin, tributyl citrate, triethyl citrate, acetyl tri-n-butyl citrate, diethyl phthalate, polyethylene glycols, castor oil, dibutyl sebacate, monoacetylated and diacetylated glycerides (e.g., Myvacet® 9-45), and mixtures thereof. When used in an embodiment of the present invention, the plasticizer may constitute from about 3% to about 30% by weight of the water-insoluble polymer. In another embodiment, the plasticizer constitutes from 10% to about 25% by weight of the water-insoluble polymer. In still other embodiments, the amount of plasticizer relative to the weight of the water-insoluble polymer is about 3%, about 5%, about 7%, about 10%, about 12%, about 15%, about 17%, about 20%, about 22%, about 25%, about 27%, and about 30%, inclusive of all ranges and subranges therebetween. One of ordinary skill in the art would know to select the type of plasticizer based on the polymer or polymers and nature of the coating system (e.g., aqueous or solvent-based, solution or dispersion-based and the total solids).

In some embodiments, the controlled-release coating disposed on the alkaline buffer layer comprises a water-insoluble polymer in combination with a water-soluble polymer and provides sustained release of melperone. In one embodiment, the ratio of the water-insoluble polymer to the water-soluble polymer ranges from about 95/5 to about 50/50, including the range of about 90/10 to about 60/40. In one embodiment, the water-insoluble and water-soluble polymers in combination constitute from about 3% to about 50% by weight of the coated bead, including the ranges from about 10% to about 50%, about 3% to about 30%, and from about 5% to about 30%. In other embodiments, the amount of water-insoluble and water-soluble polymers in combination constitute about 3%, about 5%, about 7%, about 10%, about 12%, about 15%, about 17%, about 20%, about 22%, about 25%, about 27%, about 30%, about 35%, about 40%, about 45%, and about 50% of the weight of the coated core, inclusive of all ranges and subranges therebetween.

The water-soluble polymers used in accordance with certain embodiments of the present invention encompass water-soluble polymers. Non-limiting examples of suitable water-soluble polymers include polyvinylpyrrolidone (e.g., Povidone K-25), polyethylene glycol (e.g., PEG 400), hydroxypropyl methylcellulose, and hydroxypropylcellulose. In one embodiment, the sustained-release coating provides a drug release sustained over about 12 to about 16 hours when tested in the two-stage dissolution method (700 mL of 0.1N HCl (hydrochloric acid) for the first 2 hours and thereafter in 900 mL at pH 6.8 obtained by adding 200 mL of a pH modifier), suitable for a once- or twice-daily dosing regimen.

In one embodiment, the pharmaceutical composition comprises a plurality of controlled-release particles, wherein at least a portion of the controlled-release particles comprise: (a) a core comprising melperone hydrochloride; (b) an alkaline buffer layer of disodium phosphate disposed over the core; and (c) a controlled-release coating disposed over the buffer layer, comprising ethylcellulose.

In another embodiment, the controlled-release coating comprises a water-insoluble polymer in combination with an enteric polymer, thereby providing a delayed or a timed, pulsatile release (TPR) of melperone. This type of controlled-release coating (i.e., the combination of water-insoluble and enteric polymers) may be referred to herein as a “lag-time” coating, and the microparticles coated with the lag-time coating may be referred to herein as TPR microparticles. The term “lag-time” refers to a time period wherein less than about 10% of the melperone is released from the microparticle after ingestion of the dosage form or after exposure to simulated body fluid(s). In one embodiment, the lag-time coating is deposited directly onto the alkaline buffer layer. In another embodiment, a lag-time coating is deposited directly onto one or more layers (e.g., a sealant layer) coated onto the alkaline buffer layer. In some embodiments, the ratio of the water-insoluble polymer to enteric polymer ranges from about 10:1 to about 1:4, including the ranges of from about 9:1 to about 1:3 and from about 3:1 to about 1:1. In other embodiments, the water-insoluble and enteric polymers in combination constitute from about 5% to about 60% by weight of the coated core, including the ranges of from about 10% to about 60%, and from about 10% to about 50%.

Non-limiting examples of suitable enteric polymers include cellulose acetate phthalate, hydroxypropyl methylcellulose phthalate, hydroxypropyl methylcellulose acetate succinate, polyvinyl acetate phthalate, pH-sensitive methacrylic acid-methylmethacrylate copolymers, shellac, and mixtures thereof. (The term “pH sensitive” refers to polymers which exhibit pH dependent solubility.) These enteric polymers may be used as a solution in a solvent mixture or an aqueous dispersion. Some commercially available materials that may be used are methacrylic acid copolymers sold under the trademark Eudragit (L100, 5100, L30D) manufactured by Rohm Pharma, Cellacefate (cellulose acetate phthalate) from Eastman Chemical Co., Aquateric (cellulose acetate phthalate aqueous dispersion) from FMC Corp., and Aqoat (hydroxypropyl methylcellulose acetate succinate aqueous dispersion) from Shin Etsu K.K. In one embodiment, the TPR-coating comprises ethylcellulose (e.g., EC-10) as the water-insoluble polymer and hypromellose phthalate (e.g., HP-55) as the enteric polymer.

In one embodiment, the TPR microparticles provide a lag time of from about 1 hour to about 10 hours, including from about 2 hours to about 7 hours, from about 2 hours to about 4 hours (“shorter lag time”), and from about 7 hours to about 8 hours (“longer lag time”). In another embodiment, the TPR microparticles release melperone over a period of about 4 hours to about 16 hours in the gastrointestinal tract after a lag time of about 1 hour to about 10 hours following oral administration.

In another embodiment, the microparticles contain an outer, lag-time coating disposed on a sustained-release coating. This type of embodiment begins to release melperone in the higher pH of the intestine, followed by sustained-release of melperone.

The melperone release profiles of SR and TPR microparticles may be determined by dissolution testing in a USP Apparatus 1 or 2 using a two-stage dissolution medium (first 2 hours in 700 mL of 0.1N HCl at 37° C. followed by dissolution testing at pH 6.8 obtained by the addition of 200 mL of a pH modifier). Melperone release over time can be determined various methods, for example by HPLC on samples pulled at selected time points.

The SR or TPR coating contributes to the control of melperone dissolution at the drug interface and hence melperone release from the microparticles. The achievable lag time or sustained-release time depends on the composition and thickness of the sustained-release coating, and/or the composition and thickness of the lag-time coating. Specific factors that can affect achieving optimal once-daily dosage forms include, but are not limited to, the melperone's pKa, and its solubility, e.g. in the alkaline pH microenvironment created by the alkaline buffer layer.

In another embodiment, the microparticles contain a compressible coating disposed on the controlled-release coating (or disposed on the outer-most coating, if the controlled-release coating is further coated with a TPR coating). The compressible coating comprises a polymer, for example selected from the group consisting of hydroxypropylcellulose, poly(vinyl acetate-vinyl pyrrolidone), polyvinyl acetate, and plasticized low-viscosity ethylcellulose latex dispersion. This coating may be applied, for example, by fluid-bed coating with a plasticized aqueous dispersion of ethylcellulose. The function of the compressible coating is to maintain membrane integrity during compression with rapidly dispersing microgranules.

The microparticle core comprises melperone. In some embodiments, the core can take the form of an inert bead, a microgranule, or a melperone crystal. In one embodiment, the core comprises an inert bead, coated with a layer comprising melperone or a pharmaceutically acceptable salt, solvate, and/or ester thereof. The inert bead can comprise sugar, microcrystalline cellulose, mannitol-microcrystalline cellulose, silicon dioxide, etc. The core has an average particle size of not more than 400 μm, or, in another embodiment, not more than 350 μm. In one embodiment, the microparticle core comprises an inert beat coated with a melperone layer, wherein the melperone layer further comprises a polymeric binder. The polymeric binder can be selected from the group consisting of hydroxypropylcellulose, povidone, methylcellulose, hydroxypropyl methylcellulose, carboxyalkylcellulose, polyethylene oxide, starch (e.g., corn starch or gelatinized corn starch), and a polysaccharide. The ratio of melperone to the polymeric binder can range from about 85:15 to about 100:0 (no binder).

The pharmaceutical compositions described herein can further comprise rapidly dispersing granules comprising a saccharide and/or a sugar alcohol in combination with a disintegrant. Suitable disintegrants include, for example, disintegrants selected from the group consisting of crospovidone, sodium starch glycolate, crosslinked sodium carboxymethylcellulose, low-substituted hydroxypropylcellulose, and combinations thereof. Suitable saccharides and/or sugar alcohols may be selected from the group consisting of lactose, sucralose, sucrose, maltose, mannitol, sorbitol, xylitol, maltitol, and combinations thereof. The ratio of the disintegrant to the saccharide and/or sugar alcohol in the rapidly dispersing microgranules ranges from about 1/99 to about 10/90, and in some embodiments is about 5/95 (by weight). In some embodiments, the disintegrant or the saccharide and/or sugar alcohol, or both, can be present in the form of microparticles having an average particle size of about 30 μm or less. The ratio of the melperone-containing microparticles to the rapidly disintegrating granules can range from about 1:6 to about 1:2.

In another embodiment, the present invention relates to pharmaceutical dosage forms comprising the microparticles described herein. The pharmaceutical dosage forms include orally disintegrating tablets (ODTs), conventional tablets (e.g., including rapidly dispersing tablets), and capsules. When the pharmaceutical dosage form is a conventional tablet, the conventional tablet comprises microparticles of the present invention, combined as needed with any pharmaceutically acceptable excipient(s), such as a polymeric binder, a disintegrant, fillers, diluents, compression aids, a lubricant, etc. When the pharmaceutical dosage form is a capsule, the capsule is filled with at least one population of microparticles of the present invention. The capsule can be a gelatin capsule, an HPMC capsule, etc.

When the pharmaceutical dosage form takes the form of an ODT, the ODT substantially disintegrates within about 60 seconds after contact with saliva in the oral cavity or with simulated saliva fluid. In another embodiment, the ODT substantially disintegrates within about 30 seconds. Disintegration is tested according to the USP <701> Disintegration Test. In one embodiment, the ODT comprises a therapeutically effective amount of melperone, wherein after administration the ODT substantially disintegrates in the oral cavity of a patient forming a smooth, easy-to-swallow suspension having no gritty mouthfeel or aftertaste and provides a target PK profile (i.e., plasma concentration vs. time plot) of melperone suitable for a once- or twice-daily dosing regimen.

When the pharmaceutical dosage form is a tablet, it preferably has a friability of less than about 1%. When the dosage form is an ODT, the ODT may also include pharmaceutically acceptable excipients typically used in disintegrating tablet formulations such as compressible diluents, fillers, coloring agents, and optionally a lubricant.

The ODTs comprise the one or more populations of SR or TPR microparticles described here, or mixtures thereof, combined with rapidly disintegrating microparticles. The ODTs may further comprise IR particles, preferably taste-masked to achieve acceptable organoleptic properties, i.e., smooth, non-gritty mouthfeel and no aftertaste. For example, the pharmaceutical dosage form may comprise: SR microparticles in combination with rapidly dispersing granules (e.g., mannitol-crospovidone microgranules); TPR microparticles in combination with rapidly dispersing granules; IR microparticles, SR microparticles, and rapidly dispersing granules; taste-masked IR microparticles, TPR microparticles, and rapidly dispersing granules; or taste-masked IR microparticles, SR microparticles, and one or more populations of TPR microparticles which may have the same or different lag times (e.g., short lag-time TPR microparticles and long lag-time TPR microparticles), combined with rapidly dispersing granules. These different combinations of microparticles can achieve different desired melperone release profiles. For example, a once-daily dosage form of melperone with an elimination half-life of about 7 hours may contain a mixture of an IR bead population which provides an immediate-release pulse, a second SR bead or TPR bead population with a shorter lag time (about 2-4 hours), which provides a rapid sustained-release profile, and a third TPR bead population with a longer lag time (about 7-8 hours), which allows typically a delayed, sustained-release profile over about 8-12 hours, to maintain acceptable plasma concentrations at 12-24 hours.

When IR particles are present in the pharmaceutical dosage form, the ratio of IR particles to SR and/or TPR particles ranges from about 0:100 (no IR particles) to about 50:50. The IR particles may be taste-masked by applying a taste-masking layer that substantially masks the taste of melperone contained in the particle. These taste-masked IR particles release not more than about 10% of the melperone contained in the IR particles in 3 minutes (the longest typical residence time anticipated for the ODT in the buccal cavity) when dissolution tested in simulated saliva fluid (pH ˜6.8) while releasing not less than about 75% of the melperone contained in the IR particles in about 60 minutes when dissolution tested in 0.1N HCl.

The IR particles comprise a melperone-containing core optionally coated with water-insoluble polymer (e.g., ethylcellulose), providing a taste-masking layer. If present, the coating of water-insoluble polymer on the IR particles may further comprise a plasticizer. It can further comprise a gastrosoluble pore-former (e.g., calcium carbonate), for example in accordance with the disclosure in the co-pending U.S. patent application Ser. No. 11/213,266 filed Aug. 26, 2005 (Publication No. U.S. 2006/0105038 published May 18, 2006) or by fluid-bed coating with a water-insoluble polymer (e.g., ethylcellulose with a mean viscosity of 10 cps) alone or in combination with a gastrosoluble polymer (e.g., Eudragit E100 or EPO), for example in accordance with the disclosure in the co-pending U.S. patent application Ser. No. 11/248,596 filed Oct. 12, 2005 (Publication No. U.S. 2006/0078614 published Apr. 13, 2006) or a gastrosoluble pore-former (e.g., calcium carbonate), for example in accordance with the disclosure in the co-pending U.S. patent application Ser. No. 11/256,653 filed Oct. 21, 2005 (Publication No. U.S. 2006/0105039 published May 18, 2006). Each of these applications set forth herein are incorporated by reference in their entirety for all purposes.

The ODTs described herein can have one or more of the following properties or compositions: (i) disintegrating on contact with saliva in the oral cavity in about 60 seconds, forming a smooth, easy-to-swallow suspension comprising taste-masked melperone-containing particles; (ii) disintegrating within about 30 seconds when tested by the USP <701> Disintegration Test; (iii) taste-masked IR particles, if present, provide rapid, substantially complete release of the melperone dose upon entry into the stomach (e.g., typically greater than about 75% in about 60 minutes); and/or (iv) a melperone-containing SR and/or TPR particles which sustain and/or delay release of melperone in the gastrointestinal tract.

When the pharmaceutical dosage form is a conventional tablet (for example, a rapidly dispersing tablet), one or more populations of IR, SR or TPR microparticles can be combined with appropriate pharmaceutically acceptable excipients (for example, a binder, a diluent/filler, and a disintegrant) to produce conventional tablets. These conventional rapidly dispersing tablets rapidly disperse on entry into the stomach (or disperse in a glass of water to provide an alternate mode of administration in patients who experience difficulty in swallowing tablets or capsules). Alternative dosage forms such as sprinkle capsules or sachets, comprising one or more populations of IR, SR or TPR microparticles exhibiting a smooth (non-gritty) mouthfeel and no aftertaste upon oral administration, are also embodiments of dosage forms of the present invention.

In another embodiment, the present invention is directed to methods of preparing a pharmaceutical composition of the microparticles described herein. In one embodiment, the method comprises: (a) preparing a core comprising melperone; (b) coating the melperone-containing core of step (a) with a layer comprising an alkaline buffer; and (c) coating the alkaline-buffer layered core of step (b) with a controlled-release layer. The step of preparing the core may be accomplished by any of the methods known in the art; for example, layering an inert bead (e.g., sugar, microcrystalline cellulose, mannitol-microcrystalline cellulose, silicon dioxide, etc.) with a solution comprising the drug and optionally a polymeric binder (e.g., by fluid-bed or pan coating); granulating the drug with an appropriate diluent (e.g., microcrystalline cellulose); extruding and spheronizing the drug mixture; compressing the drug into mini-tablets of about 1-2 mm in diameter; or simply obtaining drug crystals of the desired particle size (e.g., about 50-500 μm, including 100-400 μm).

In another embodiment, the method further comprises coating the melperone-containing core with a sealant layer before coating with a layer comprising an alkaline buffer.

In one embodiment, the method is used to prepare a microparticle with a sustained-release coating. In this embodiment, the controlled-release coating of step (d) comprises a water-insoluble polymer and optionally a water-soluble polymer for a weight gain of from about 3% to about 40% to give a SR microparticle. In another embodiment, the method is used to prepare microparticles with a timed, pulsatile release (TPR) coating, or lag-time coating. In this embodiment, the controlled-release coating of step (d) comprises a water-insoluble polymer and an enteric polymer for a weight gain of from about 10% to about 60% to give a TPR microparticle. In another embodiment, the method is used to prepare microparticles with a sustained-release coating underlying an outer timed-pulsatile release coating. In this embodiment, the controlled-release coating of step (d) comprises a water-insoluble polymer and optionally a water-soluble polymer for a weight gain of from about 3% to about 30% to give a sustained-release microparticle. This sustained-release microparticle is further coated with a layer comprising a water-insoluble polymer and an enteric polymer to give a TPR microparticle.

In another embodiment, the present invention relates to a method of preparing a pharmaceutical dosage form comprising: mixing the microparticles described herein with rapidly dispersing granules comprising a saccharide and/or sugar alcohol in combination with a disintegrant; and compressing the resulting mixture into a tablet to provide an ODT. In still another embodiment, the pharmaceutical dosage form may be prepared by filling a capsule (e.g. a hard-gelatin capsule) with the microparticles described herein.

In one embodiment, the method comprises the steps of:
a) preparing melperone particles (crystals, microgranules, beads, or pellets with an average particle size of 50-500 μm, more particularly of 100-400 μm) and applying a protective seal-coat onto the melperone-layered particles to produce IR particles;
b) applying an alkaline buffer layer onto the IR particles (optionally from a solution of further comprising a polymeric binder) and applying a protective seal-coat onto the buffer layer;
c) applying a sustained-release coating comprising a water-insoluble polymer or a water-insoluble polymer in combination with a water-soluble polymer for a weight gain of from about 3% to 30% to produce SR particles; and/or
d) applying a lag-time coating onto SR particles or alkaline buffer layered particles a combination of water-insoluble and enteric polymers at a weight ratio of from about 10:1 to 1:4 for a weight gain of from about 10% to 60% by weight of the coated bead to produce TPR particles; and
e) filling the IR, SR and/or TPR particles into hard-gelatin capsules; or compressing the SR or TPR particles (optionally in combination with an additional pharmaceutically acceptable excipients) into conventional tablets or orally disintegrating tablets (ODTs). The combination of IR beads, SR beads and/or TPR beads can be combined at a ratio which provides the desired melperone release profile.

The following non-limiting examples illustrate the melperone dosage forms of the present invention, as capsules, conventional tablets or orally disintegrating tablets comprising an immediate release bead population and/or one or more SR or TPR bead populations, each with a predetermined onset, and in which the totality of the in vitro drug-release profile or, upon oral administration, the in vivo plasma concentration profile of the dosage form provides maximum therapeutic efficacy, and enhances patient compliance and quality of life. Such dosage forms, when administered appropriately, maintain melperone plasma concentrations at levels which minimize the occurrence of side-effects associated with Cₘₐₓ or Cₘᵢₙ.

### EXAMPLES

### Example 1

### Deconvoluted In Vitro Release Profiles for Melperone QD

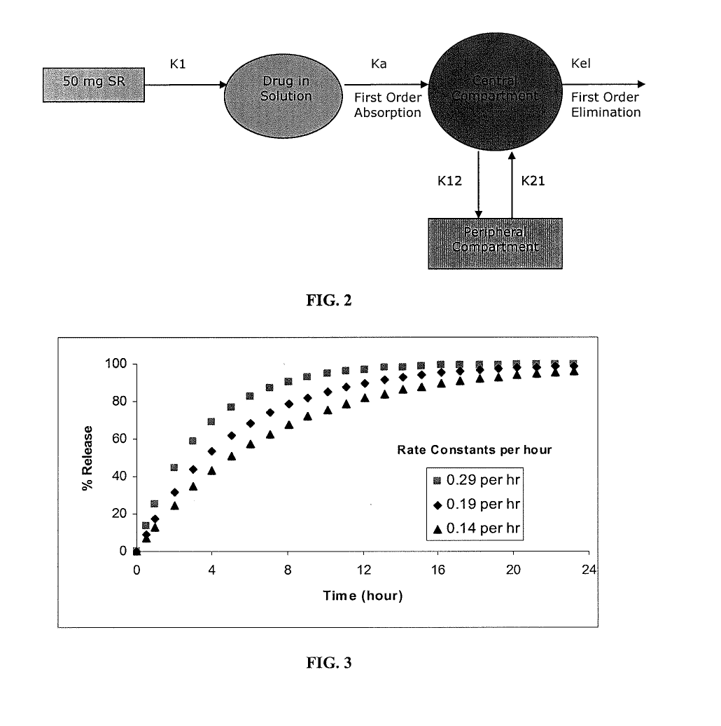

A pharmacokinetic evaluation was undertaken to identify a set of theoretical in vitro melperone-release profiles that would be suitable for a once- or twice-daily dosage form of melperone. Using human plasma concentration-time data upon oral single dose administration or at steady state and/or upon an intravenous (IV) profile, melperone was found to fit a two-compartment model (see Table 1, below, and FIG. 2). Oral data (PO) shows the absence of a well-defined distribution phase, but both IV and PO data could be fit simultaneously to the two-compartment model shown below. Using WinNonlin Software, PK parameter estimates and predictions of both PO and IV data were performed as shown in Table 1. From these estimates, equations for simulated profiles were generated and are shown in FIG. 3. Rate constants 0.29, 0.19, and 0.14 per hour are fast CR, medium CR, and slow CR, respectively. Formulations with in vitro drug-release profiles that mimicked the simulated profiles of FIG. 3 were developed and tested in a pilot PK study in adult healthy subjects.

TABLE 1
Parameter estimates of 20 mg IV and 50 mg Peroral Melperone

| | Estimate | Std Error | CV % |
|---|---|---|---|
| Volume | 205406 | 4941.2 | 2.41 |
| Kₐ | 0.758 | 0.136 | 18.00 |
| K₁₀ | 0.935 | 0.053 | 5.69 |
| K₁₂ | 4.730 | 0.207 | 4.37 |
| K₂₁ | 1.358 | 0.073 | 5.35 |
| α | 6.838 | 0.277 | 4.05 |
| β | 0.186 | 0.014 | 7.57 |
| A | 80.205 | 2.188 | 2.73 |
| B | 17.164 | 0.598 | 3.49 |
| T_{lag} | 0.448 | 0.021 | 4.62 |

### Example 2

### 2.A Melperone Hydrochloride IR Beads at a Drug Load of 25%

Hydroxypropyl cellulose (Klucel® LF, 42.9 g) was slowly added to ethanol (357.2 g) until dissolved under constant stirring for not less than 10 min, and then water (100 g) was mixed into the resulting solution. Melperone hydrochloride (obtained from Lundbeck) (357.2 g) was slowly added to 3114.8 g of purified water until dissolved. Then the Klucel® solution was added to the melperone hydrochloride solution, and mixed for 10 min. A Glatt GPCG 3 equipped with a 7″ bottom spray Wurster 7 13/16″ column, ‘C’ bottom air distribution plate covered with a 200 mesh product retention screen (1.0 mm port nozzle) was charged with 1000 g of 60-80 mesh sugar spheres and sprayed with the melperone/Klucel® solution at an initial rate of 2 mL/min with a stepwise increase to 18 mL/min, at an inlet air volume set at 50 CFM, air atomization pressure of 1.25 bar while maintaining the product temperature of 36±3° C. Following rinsing of the spray system with 50 g of acetone, a seal-coat solution of Klucel® LF dissolved in 85/15 acetone/water, and magnesium stearate suspended in the Klucel® solution (7.0% solids) was sprayed at 18 mL/min, and the seal-coated beads were dried in the Glatt unit for 5 min to drive off residual solvents (including moisture). The resulting melperone IR beads (Lot# 1272-038) were sieved through 35 and 80 mesh screens to discard oversized particles and fines.

### 2.B Melperone Hydrochloride IR Beads (Drug Load: 25%)

Melperone hydrochloride (obtained from Lundbeck) (1027.4 g) was slowly added to 50/50 acetone/purified water (3424.7 g each) until dissolved, while stirring constantly. A Glatt GPCG 5 equipped with a 9″ bottom spray Wurster 14″ column, ‘D’ bottom air distribution plate covered with a 200 mesh product retention screen, 1.0 mm port nozzle, was charged with 3000 g of 60-80 mesh sugar spheres and the sugar spheres were sprayed with the melperone solution at an initial rate of 10 mL/min with a stepwise increase to 30 mL/min, at an inlet air volume set at 100 CFM, air atomization pressure of 1.4 bar while maintaining the product temperature of 33±3° C. Following rinsing of the spray system with 50 g of acetone, a seal-coat solution (Klucel® LF dissolved in 85/15 acetone/purified water at 7% solids) was sprayed at 10-15 mL/min at the product temperature of 41° C. for a weight gain of 2% and dried in the Glatt unit for 5 min to drive off residual solvents (including moisture). The resulting IR beads (Lot# 1272-070) were sieved through 35 and 80 mesh screens to discard oversized particles and fines.

Two different batches of IR beads, with and without the incorporation of a polymer binder (Klucel® LF) were thus prepared, as disclosed in 2.A and 2.B, above. However, it was determined that for melperone hydrochloride coated beads, the polymeric binder was not needed. Accordingly, in the succeeding examples, the melperone hydrochloride layer does not include a polymeric binder.

### 2.C Disodium Phosphate Anhydrous (DPA) Buffer Layering

Anhydrous disodium phosphate (42.9 g) was added with stirring to purified water (858 g) until dissolved. A Glatt GPCG 3 equipped with a 6″ bottom spray Wurster 8″ column 13 and ‘C’ distribution plate covered with a 200 mesh product retention screen and 1.0 mm port size nozzle, was charged with IR beads (1301 g) from above (i.e., IR beads according to 2.B, above). The disodium phosphate solution was sprayed onto the IR beads while fluidizing at an inlet air volume of 20 CFM, target product temperature 45±2° C. at a spray rate of 2 mL/min with a stepwise increase to 20 mL/min for a weight gain of 3.12%. After rinsing the spray system with acetone, a protective seal coat solution (Klucel® LF dissolved in 85/15 acetone/water, 7.0% solids) was coated onto the disodium phosphate coated beads at a coating weight of about 2% by weight (relative to the weight of the coated bead), and at a product temperature of 33±1° C. and a flow rate of 10 mL/min. The dried disodium phosphate coated IR beads (Lot# 1272-125) were sieved using 35 and 80 mesh sieves to discard oversized beads and fines.

### 2.D Melperone SR Beads (SR Coat: 30% w/w)

The alkaline buffer-coated beads (1000 g) from 2.C, above, were fluid-bed coated with an SR functional polymer coating of plasticized ethylcellulose (408.1 g ethylcellulose, 33.1 g dibutyl sebacate) dissolved in a 85/15 mixture of acetone and water (10% solids) at a product temperature of 33±1° C., atomization air pressure of 1.25 bar, inlet air volume of 15 CFM, and an initial flow rate of 8 mL/min with a stepwise increase to 20 mL/min for a SR coating level of 30% by weight. Hydroxypropylcellulose (Klucel® LF 29.4 g) dissolved in acetone (333.2 g) and water (58.8 g) was then coated onto the SR-coated beads at a spray rate of 10 mL/min at a product temperature of 33±1° C. for a weight gain of 2% by weight (relative to the weight of the seal-coated bead), to provide SR beads coated with a compressible polymer layer. The resulting SR beads were dried in the Glatt unit for 5 min to drive off residual solvents. The dried beads (Lot# 1272-127) were sieved using 30 and 80 mesh sieves to discard oversized beads and fines.

### 2.E Rapidly Dispersing Microgranules

Rapidly dispersing microgranules were prepared following the procedure disclosed in co-pending US Patent Application Publication No. U.S. 2005/0232988, published Oct. 20, 2005, the contents of which are hereby incorporated by reference in their entirety for all purposes. Specifically, D-mannitol (152 kg) with an average particle size of approximately 20 μm or less (Pearlitol 25 from Roquette, France) was blended with 8 kg of cross-linked povidone (Crospovidone XL-10 from ISP) in a high shear granulator (GMX 600 from Vector), granulated with purified water (approximately 32 kg), wet-milled using a Comil from Quadro, and finally tray-dried to provide microgranules having an LOD (loss on drying) of less than about 0.8%. The dried granules were sieved and oversize material was again milled to produce rapidly dispersing microgranules with an average particle size in the range of approximately 175-300 μm.

### 2.F Melperone HCl CR ODT Comprising SR Beads (50 mg)

Rapidly dispersing microgranules (1235.1 g) prepared in 2.E were blended with melperone HCl SR beads (856.2 g) and peppermint flavor (25 g), sweetener (8.8 g sucralose), additional crospovidone (125 g), and Avicel PH101 (250 g microcrystalline cellulose) at a ratio of rapidly dispersing microgranules to multicoated Melperone HCl beads of about 3:2 in a twin shell V-blender for a sufficient time to provide a homogeneously distributed blend for compression. ODTs comprising 50 mg melperone HCl as SR beads were compressed using a production scale tablet press equipped with an external lubrication system at the following conditions: tooling: 15 mm round, flat face, radius edge; compression force: 16 kN; mean weight: 1000 mg; mean hardness: 46 N; and friability: 0.28%. Melperone HCl ODT CR, 50 mg thus produced rapidly disintegrate in the oral cavity creating a smooth, easy-to-swallow suspension comprising coated melperone HCl beads, and provide a target profile suitable for a once-daily dosing regimen.

### 2.G Melperone SR Beads

IR beads (Lot# 1295-189) containing melperone hydrochloride (drug load: 21% w/w) were prepared in the Glatt GPCG 3 as disclosed in 2.B and were further coated with DPA as disclosed in 2.0 to produce DPA coated IR beads, Lot# 1295-191 at a DPA load of about 10%. The DPA coated IR beads (Lot# 1295-191) were further coated with EC-10/DBS at a ratio of 92.5/7.5 for a weight gain of about 30% by weight as disclosed in 2.D, above to produce SR beads (Lot# 1348-004).

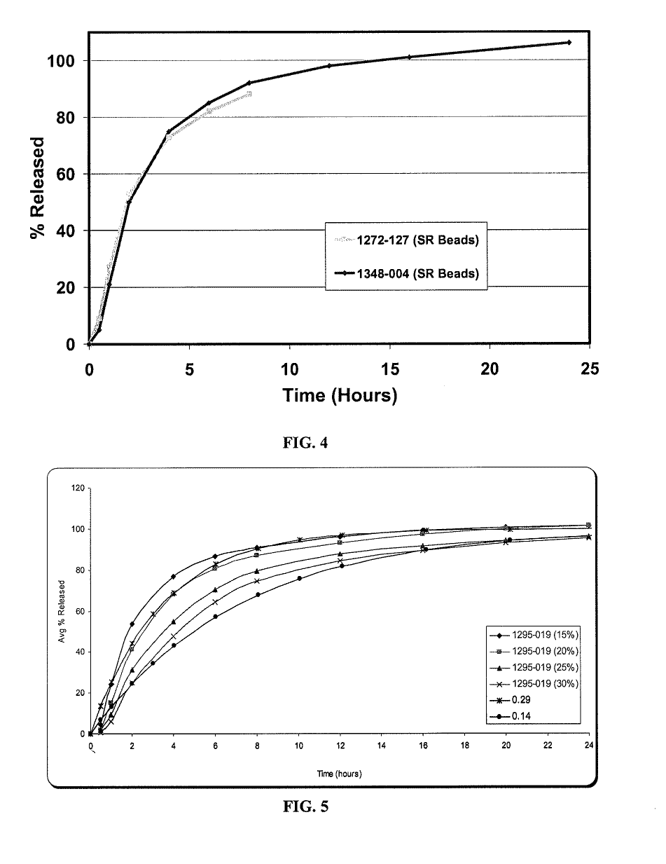

FIG. 4 shows similar release profiles of Melperone HCl for SR beads prepared as in 2.D (Lot# 1272-127 at 25% drug load and DPA coating at 3.1%) and 2.G (Lot# 1348-004 at 20% drug load and DPA coating at 10%) indicating that the amount of the melperone hydrochloride loading and alkaline buffer layer loading on the IR beads can be varied to provide similar release profiles.

### Example 3

### 3.A Melperone Hydrochloride IR Beads (Drug Load: 20% w/w)

Melperone hydrochloride (obtained from Lundbeck) (1095.9 g) was slowly added with stirring to a 50/50 mixture of acetone and water (3653 g each) until dissolved. A Glatt GPCG 5 equipped with a 9″ bottom spray Wurster 10″ column, ‘D’ bottom air distribution plate covered with a 200 mesh product retention screen, 1.0 mm port nozzle, was charged with 3200 g of 45-60 mesh sugar spheres, which were then sprayed with the melperone hydrochloride solution at an initial rate of 10 mL/min with a stepwise increase to 36 mL/min, at an inlet air volume set at 100 CFM, and an air atomization pressure of 1.40 bar, while maintaining the product temperature of 33±3° C. Following rinsing of the spray system with 50 g of acetone, a seal-coat solution (Klucel® LF dissolved in 85/15 acetone/water, 7.5% solids) was sprayed onto the melperone hydrochloride coated beads at an initial rate of 10 mL/min with a stepwise increase to 15 mL/min., and dried in the Glatt unit for 5 min. to drive off residual solvents (including moisture). The resulting IR beads were sieved to discard oversized (>500 μm or 35 mesh) beads and fines (<80 mesh).

### 3.B Melperone Buffer-Coated Beads

Dibasic sodium phosphate (461.5 g) was slowly added to 9230 g of purified water, with stirring. Melperone hydrochloride IR beads (3900 g) from 3.A, above, were charged into a Glatt GPCG 5, and coated with the aqueous alkaline buffer solution at a fluidization air volume of 90 CFM, an atomization air pressure of 1.85 bar, and target product temperature of 36° C., at an initial spray rate of about 10 mL/min with a stepwise increase to 25 mL/min. Following an acetone rinse of the spray system, a seal coat (Klucel® LF dissolved in 85/15 acetone/water, 7.5% solids) was applied to the dibasic phosphate coated beads for a weight gain of about 2% w/w at a product temperature of 33±2° C. and a flow rate of 10-15 mL/min. The seal coated beads were then dried in the Glatt unit for 10 min to drive off residual solvents.

### 3.C Melperone SR Beads (Coating: 30% w/w)

An SR coating solution was prepared by slowly adding ethylcellulose, EC-10 (408.1 g) to 3750 g of acetone while stirring until dissolved; purified water (662 g) was then added with stirring; then dibutyl sebacate (33.1 g) was slowly added to the ethylcellulose/dibutyl sebacate solution, and stirred for 10 min. The buffer coated melperone IR beads (1000 g) from 3.B, above were charged into a Glatt GPCG 3 equipped with a 6″ bottom spray Wurster 8″ column, 1.0 mm nozzle port, and a bottom ‘C’ distribution plate, and coated with the SR coating solution at a fluidization air volume of 15 CFM, atomization air pressure of 1.25 bar, target product temperature of 33° C., and an initial spray rate of about 10 mL/min with a stepwise increase to 20 mL/min. Samples were pulled at coating levels of about 15%, 20%, 25% by weight for dissolution testing. Following an acetone rinse of the spray system, a compressible coating of hydroxypropylcellulose (Klucel® LF 29.4 g) was applied as disclosed in 3.B, above to produce SR beads. FIG. 5 shows the release profiles of melperone from SR beads coated at different thicknesses. The figure also shows the theoretical slow-release (0.14 h⁻¹) and fast-release (0.29 h⁻¹) profiles for comparison.

### 3.D Melperone HCl ODT CR, 50 mg

Rapidly dispersing microgranules (1407.4 g) (prepared as described in 2E, above) were blended with the melperone hydrochloride SR beads (1102.1 g) from 3.C, above, and other pre-blended pharmaceutically acceptable ingredients (i.e., peppermint flavor (30.0 g), sweetener (10.5 g sucralose), crospovidone (150.0 g) and Avicel PH101 (300.0 g microcrystalline cellulose) at a ratio of rapidly dispersing microgranules to Melperone HCl SR beads (SR beads of 4.C, above) of about 3:2 in a twin shell V-blender for a sufficient time to provide a homogeneously distributed blend for compression. ODTs comprising 50 mg melperone HCl as TPR Beads were compressed using a production scale Hata Tablet Press equipped with an external lubrication system (Matsui ExLube System) at a mean tablet weight of 1 g and mean hardness of about 46 N. Melperone HCl ODT CR (50 mg) thus produced rapidly disintegrate in the oral cavity creating a smooth, easy-to-swallow suspension comprising coated melperone HCl beads, and which provide a target profile suitable for a once-daily dosing regimen.

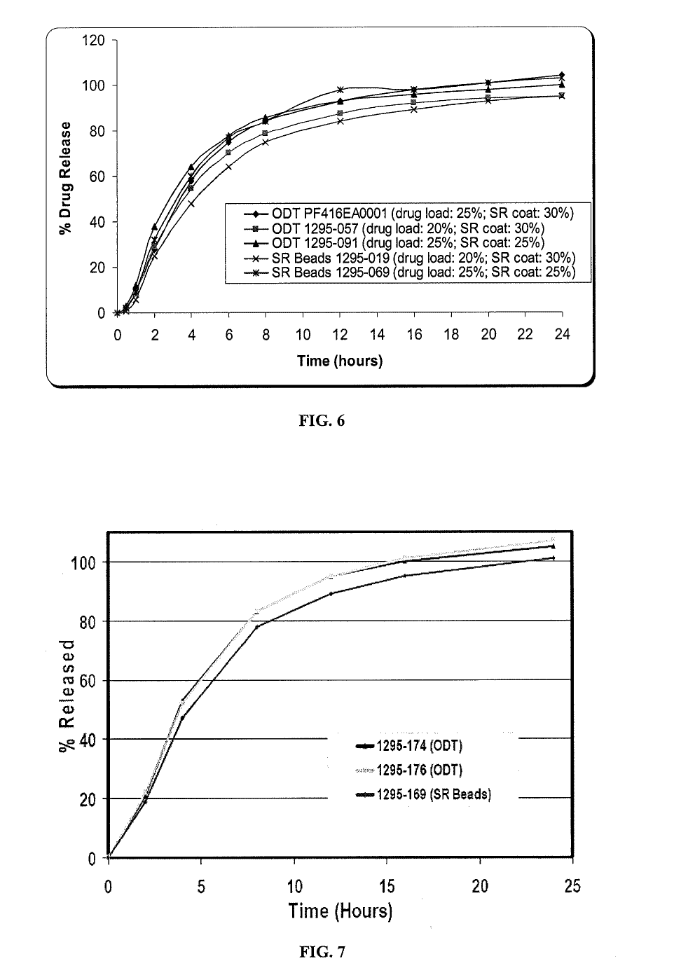

Two batches of melperone IR beads were prepared according to the procedure of 3.A (at drug loadings of 20% w/w and 25% w/w; Lot# 1295-011 & Lot# 1295-036). These IR beads were coated with DPA according to the procedure of 3.B (Lot# 1295-019 & Lot# 1295-067, respectively). SR beads were then prepared by coating the DPA-coated beads with an SR coating according to the procedure of 3.0 (Lot# 1295-019 & Lot# 1295-069, respectively), and ODT CR tablets were then prepared from the SR coated beads according to the procedure of 3.D (Lot# 1295-057 & Lot# 1295-091; see Table 2 for ODT CR compositions). FIG. 6 shows similar release profiles for Melperone HCl SR beads (Lot# 1295-019 & Lot# 1295-069) and the corresponding Melperone HCl ODT CR (Lot# 1295-057 & Lot# 1295-091) indicating minimal membrane fracture during tableting.

TABLE 2
Compositions and Tableting properties of ODTs

| | Compositions | | | | |
|---|---|---|---|---|---|
| | | | ODTs | | |
| | SR Beads | | | | PF416EA |
| | 1295-019 | 1295-069 | 1295-057 | 1295-091 | 0001 |
| Ingredients | %/Bead | %/Bead | %/tablet | | |
| Melperone HCl SR Beads | 100.0 | 100.0 | 39.06 | 31.25 | 36.63 |
| Compressible Coating | 2.0 | 2.0 | | | |
| SR Coating (EC-10/DBS) | 30.0 | 25.0 | | | |
| DPA Coated IR Beads | 68 (100) | 73 (100) | | | |
| Seal Coat (Klucel ® LF) | (2.0) | (2.0) | | | |
| Disodium Phosphate (DPA) | (8.5) | (10.4) | | | |
| Mel HCl IR Beads | (89.5) | (87.6) | | | |
| | [100] | [100] | | | |
| Seal Coat (Klucel ® LF) | [2.0] | [2.0] | | | |
| Melperone HCl | [20.0] | [25.0] | | | |
| Sugar Spheres (45-60) | [78.0] | [73.0] | | | |
| Rapidly Dispersing Granules | | | 44.59 | 49.40 | 47.02 |
| MCC -- Avicel PH101 | | | 10.00 | 12.50 | 10.00 |
| Crospovidone (XL-10) | | | 5.00 | 5.00 | 5.00 |
| Sucralose | | | 0.35 | 0.35 | 0.35 |
| Wintergreen (WF) or | | | 1.0 (PF) | 1.5 (WF) | 1.0 (PF) |
| Peppermint Flavor (PF) | | | | | |
| Magnesium Stearate | | | Trace | Trace | Trace |
| Tablet Weight (mg) | | | 1000 | 1000 | 1000 |

* % of the ingredients in the DPA coated IR beads are given in ( ) while the % of the ingredients in the IR beads are given in [ ].

### Example 4

### Melperone HCl ODT CR, PF416EA0001

ODT CR (Lot# PF416EA0001) comprising melperone HCl SR beads was prepared according to the methods of 3.A-3.D (SR coating: 92.5/7.5 EC-10/DBS at 30% w/w disposed on DPA buffer layered IR beads at a drug load of 25% w/w disposed on 45-60 mesh sugar spheres (see Table 2 for the composition and FIG. 6 for the in vitro drug release profile of PF416EA0001).

### Example 5

### Melperone TPR Beads (drug load: 25% w/w; Alkaline buffer (MgO): 10% w/w; EC-10/HP-55/TEC at 40%)

Melperone hydrochloride is layered on Cellets 100 (100-200 μm microcrystalline cellulose inert cores) from an aqueous solution in Glatt GPCG 3 (e.g., equipped with a 6″ bottom spray Wurster 7 13/16″ column, ‘C’ bottom air distribution plate covered with a 200 mesh product retention screen), and the drug layered beads are provided with a protective coating with Klucel® LF at 2% w/was disclosed in Example 2.B, above. The IR beads are then coated with a dispersion of hydroxypropylcellulose (binder) and micronized magnesium oxide for a weight gain of 10% by weight and further coated with a 2% seal coat of Klucel® LF. These buffer-coated IR beads are subsequently coated with a water-insoluble polymer, ethylcellulose (EC-10), an enteric polymer, (HP-55), and TEC at a ratio of 55/30/15 for a weight gain of 40% by weight. IR beads are taste-masked by coating IR beads with EC-10 and Eudragit E100 (gastrosoluble polymer) at a ratio of 1:1 dissolved in a solvent system for a weight gain of 20%. Appropriate amounts of taste-masked IR beads and TPR beads are blended with rapidly dispersing granules from 2.E, above and a pre-mix comprising microcrystalline cellulose (Ceolus KG-802 at 10% w/w), crospovidone (5% w/w), sucralose (0.35% w/w), and peppermint flavor (1.0% w/w), and compressed into dose proportional ODT tablets comprising 25-mg, 50-mg, 75-mg, and 100-mg melperone hydrochloride.

### Example 6

### 6.A Melperone IR Beads at Pivotal Scale (Drug Load: 25% w/w)

Melperone hydrochloride (15.0 kg) (obtained from Lundbeck) was slowly added to a 50/50 mixture of acetone and water (50 kg each), with stirring until dissolved. A Glatt GPCG 120 equipped with an 18″ bottom spray Wurster 23.75″ column; outer “G” and inner “C” bottom air distribution plate covered with a 100 mesh product retention screen, partition height: 50 mm; nozzle port size: 3.0 mm with HS Collar; was charged with 43.8 kg of 45-60 mesh sugar spheres. The above melperone solution was sprayed onto the sugar spheres at an initial rate of 100 g/min with ramp up (range: 75-700 g/min); inlet air volume: 450 CFM (range: 500-900 CFM); set process air temperature: 75° C. (range: 39-65° C.); atomization air pressure: 2 bar; target product temperature: 32° C. (range: 29-36° C.). Following rinsing of the spray system with 50/50 acetone/water, a sealant solution (Klucel® LF dissolved in 85/15 acetone/water, 7.5% solids) was sprayed onto the melperone-coated beads at an initial rate of 200 g/min and a stepwise increase to 300 g/min, and process air temperature of 45° C. (range: 35-50° C.). The resulting sealant-coated beads (Klucel® LF coating at 2% w/w) were then dried in the Glatt unit for 5 min. to drive off residual solvents (including moisture). The resulting IR beads were sieved to discard oversized (>500 μm or 35 mesh) beads and fines (<80 mesh).

### 6.B Melperone Buffer-Coated Beads

Dibasic sodium phosphate (6.2 kg) was slowly added to 124 kg of purified water while stirring. Melperone hydrochloride IR beads (52.6 kg) from 6.A above were charged into a Glatt GPCG 120 and coated with the aqueous alkaline buffer solution at a fluidization air volume of 650 CFM (range: 400-800 CFM); atomization air pressure: 2.5 bar, target product temperature: 53° C. (range: 49-60° C.); spray rate: 100 g/min with ramp up to 400 g/min (range: 75-700 g/min). Following the buffer layering, a protective seal coat with an Opadry Clear aqueous solution (6% solids) was applied to the DPA coated beads for a weight gain of about 2% (relative to the weight of the seal-coated beads) at a product temperature of 50° C. and a flow rate of 200 g/min (range: 100-350 g/min). The seal-coated beads were then dried in the Glatt unit for not more than 5 min. to drive off residual solvents.

### 6.C Melperone SR Beads

Ethylcellulose, EC-10 (13.9 kg) was slowly added to a 85/15 acetone/water mixture (214.2 kg), with stirring, until dissolved. Then dibutyl sebacate (1.1 kg) was slowly added to the polymer solution, and stirred for 30 min. The buffer-coated melperone IR beads (34.0 kg) from 6.B above were charged into a Glatt GPCG 120 equipped with an 18″ bottom spray Wurster 18″ column, 3.0 mm nozzle port with HS Collar and bottom inner “G” and outer “C” distribution plate, and coated with the above SR coating solution (7% solids) at an initial rate of 100 g/min with ramp up to 500 g/min (range: 75-700 g/min); inlet air volume: 500 CFM (range: 400-900 CFM); process air temperature: 64° C. (range: 40-70° C.); atomization air pressure: 2.5 bar; target product temperature: 33° C. (range: 29-40° C.). Following rinsing with acetone, the SR-coated beads were then sprayed with a seal-coat solution (Klucel® LF dissolved in 85/15 acetone/water, 7.5% solids) at an initial rate of 200 g/min (range: 100-350 g/min); process air temperature: 45° C. (range: 35-50° C.); product temperature: 32° C. (range: 28-40° C.), and then dried in the Glatt unit for 5 min. to drive off residual solvents (including moisture).

### 6.D Melperone HCl ODT CR, 50 mg

Pharmaceutically acceptable ingredients (i.e., peppermint flavor, sucralose, crospovidone, and microcrystalline cellulose; see Table 3) were first blended in a V blender to achieve a homogeneously blended pre-mix. Rapidly dispersing microgranules (prepared as described in Example 2.E) were blended with the melperone HCl SR beads (6.C) and the pre-mix in a twin shell V-blender for sufficient time to obtain a homogeneously blended compression mix. ODTs comprising 50 mg melperone HCl as SR Beads were compressed using a production scale Hata tablet press equipped with an external lubrication system (Matsui Ex-Lube System) at under tableting conditions optimized to provide the tableting properties of Table 3. Melperone HCl ODT CR, 50 mg thus produced rapidly disintegrate in the oral cavity creating a smooth, easy-to-swallow suspension comprising coated melperone HCl beads, having a release profile suitable for a once-daily dosing regimen.

Two ODT batches were produced as described above, having different tablet weights and relative amounts of melperone SR beads and rapidly dispersing granules (see Table 3). Tablets containing a higher amount of the rapidly dispersing granules had a marginally better mouthfeel and shorter disintegration time. FIG. 7 shows the drug-release profiles of Melperone HCl SR beads (Lot# 1295-169) and Melperone HCl ODT CR (1295-174 & 1295-176).

TABLE 3
Compositions and Tableting properties of ODTs

| | ODT Lot# 1295-174 | | | ODT Lot# 1295-176 | | |
|---|---|---|---|---|---|---|
| Ingredients | % per tablet | Qty/batch | | % per tablet | Qty/batch | |
| Melperone SR beads | 35.71 | 3571.4 | g | 29.76 | 2976.2 | g |
| Rapidly Dispersing | 42.94 | 4293.6 | g | 48.64 | 4863.8 | g |
| Granules | | | | | | |
| MCC - Ceolus KG-802 | 7.50 | 750.0 | g | 7.50 | 750.0 | g |
| MCC -- Avicel PH101 | 7.50 | 750.0 | g | 7.50 | 750.0 | g |
| Crospovidone (XL-10) | 5.00 | 500.0 | g | 5.00 | 500.0 | g |

| | | | | | |
|---|---|---|---|---|---|
| Silicone Dioxide | — | — | 0.25 | 25.0 | g |
| Sucralose | 0.35 | 35.0 | 0.35 | 35.0 | g |

| | | | | |
|---|---|---|---|---|
| Peppermint Flavor | 1.00 | 100.0 | 1.00 | 100.0 |
| Magnesium Stearate | Trace | Trace | Trace | Trace |

| Properties | ODT 1295-174 | | | ODT 1295-176 | | |
|---|---|---|---|---|---|---|
| Compression Force | 15 kN | 16 kN | 17 kN | 13 kN | 14 kN | 15 kN |
| Tablet Weight (mg) | 1013 | 1002 | 1014 | 1203 | 1202 | 1206 |
| Tablet Hardness (N) | 39 | 42 | 49 | 48 | 57 | 63 |
| Tablet Friability (%) | 0.39 | 0.34 | 0.25 | 0.56 | 0.44 | 0.40 |

These experiments demonstrate that the microparticles comprising melperone HCl, incorporating an alkaline buffer layer, optionally separated from the melperone by a sealant layer, and further coated with one or more controlled-release layer(s) significantly sustains and/or delays the release of melperone. Furthermore, the melperone remains in a stable, unaltered form in the solid dosage form.

A comparison of the initial portions of the drug release profiles of the ODT CR (comprising SR beads) with the corresponding SR beads in FIGS. 6 and 7, it is apparent that membrane integrity is largely maintained during the compression of these compressible coated SR beads.

We claim:

1. A pharmaceutical composition comprising one or more populations of controlled-release particles, wherein at least one population of controlled-release particles comprises:
(a) a core comprising melperone and/or a pharmaceutically acceptable salt, ester, or solvate thereof;
(b) an alkaline buffer layer disposed over the core; and
(c) a controlled-release coating disposed over the alkaline buffer layer, wherein the controlled-release coating comprises a water-insoluble polymer.

2. The pharmaceutical composition of claim 1, wherein melperone is present as melperone hydrochloride.

3. The pharmaceutical composition of claim 1, further comprising a compressible coating disposed on said controlled-release coating.

4. The pharmaceutical composition of claim 3, wherein said compressible coating layer comprises a hydrophilic polymer.

5. The pharmaceutical composition of claim 4, wherein said hydrophilic polymer is selected from the group consisting of hydroxypropylcellulose, poly(vinyl acetate-vinyl pyrrolidone), polyvinyl acetate, ethylcellulose, and mixtures thereof.

6. The pharmaceutical composition of claim 3, wherein said compressible coating comprises up to about 5% of said sustained-release particle.

7. The pharmaceutical composition of claim 1, wherein said water-insoluble polymer is selected from the group consisting of ethylcellulose, cellulose acetate, cellulose acetate butyrate, polyvinyl acetate, neutral methacrylic acid-methylmethacrylate copolymers, and mixtures thereof.

8. The pharmaceutical composition of claim 1, wherein the water-insoluble polymer comprises ethylcellulose.

9. The pharmaceutical composition of claim 1, where said controlled-release coating further comprises a plasticizer.

10. The pharmaceutical composition of claim 1, wherein said alkaline buffer is selected from the group consisting of sodium hydroxide, monosodium dihydrogen phosphate, disodium hydrogen phosphate, trisodium phosphate, sodium acetate, sodium carbonate or bicarbonate, monopotassium dihydrogen phosphate, dipotassium hydrogen phosphate, tripotassium phosphate, potassium acetate, potassium carbonate, potassium bicarbonate, magnesium phosphate, magnesium acetate, calcium silicate, complex magnesium aluminum metasilicates, magnesium carbonate, magnesium oxide, magnesium hydroxide, sodium silicate, and mixtures thereof.

11. The pharmaceutical composition of claim 1, wherein the ratio of said alkaline buffer to melperone ranges from about 5:1 to about 1:5.

12. The pharmaceutical composition of claim 11, wherein the ratio of said alkaline buffer to melperone ranges from about 3:1 to about 1:3.

13. The pharmaceutical composition of claim 1, wherein the controlled-release layer further comprises a water-soluble polymer.

14. The pharmaceutical composition of claim 13, wherein the weight of the controlled-release layer ranges from about 10% to about 50% of the total weight of said sustained-release particle.

15. The pharmaceutical composition of claim 13, wherein said water-soluble polymer is selected from the group consisting of povidone, polyethylene glycol, hydroxypropyl methylcellulose, and hydroxypropylcellulose.

16. The pharmaceutical composition of claim 15, wherein the ratio of said water-insoluble polymer to said water-soluble polymer ranges from about 95:5 to about 50:50.

17. The pharmaceutical composition of claim 1, wherein the controlled-release layer further comprises an enteric polymer.

18. The pharmaceutical composition of claim 1, further comprising an outer, lag-time coating disposed over said controlled-release coating.

19. The pharmaceutical composition of claim 1, wherein the core comprises an inert bead coated with melperone and/or a pharmaceutically acceptable salt, ester, or solvate thereof.

20. The pharmaceutical composition of claim 19, wherein said inert bead comprises sugar, microcrystalline cellulose, lactose, mannitol-microcrystalline cellulose, or silicon dioxide.

21. The pharmaceutical composition of claim 1, wherein said core has an average particle size of not more than about 400 μm.

22. The pharmaceutical composition of claim 19, wherein said melperone coating further comprises a polymeric binder.

23. The pharmaceutical composition of claim 22, wherein said polymeric binder is selected from the group consisting of hydroxypropylcellulose, povidone, methylcellulose, hydroxypropyl methylcellulose, carboxyalkylcellulose, polyethylene oxide, starch, and a polysaccharide.

24. The pharmaceutical composition of claim 1, wherein said alkaline buffer layer further comprises a polymeric binder.

25. The pharmaceutical composition of claim 24, wherein said polymeric binder is selected from the group consisting of hydroxypropylcellulose, povidone, methylcellulose, hydroxypropyl methylcellulose, carboxyalkylcellulose, polyethylene oxide, starch and a polysaccharide.

26. The pharmaceutical composition of claim 1, wherein said composition further comprises rapidly dispersing granules comprising a saccharide and/or sugar alcohol in combination with a disintegrant.

27. The pharmaceutical composition of claim 26, wherein said disintegrant is selected from the group consisting of crospovidone, sodium starch glycolate, crosslinked sodium carboxymethyl cellulose, and low-substituted hydroxypropylcellulose.

28. The pharmaceutical composition of claim 26, wherein said saccharide and/or sugar alcohol is selected from the group consisting of sucralose, lactose, sucrose, maltose, mannitol, sorbitol, xylitol, and maltitol.

29. The pharmaceutical composition of claim 26, wherein the ratio of said disintegrant to said saccharide and/or sugar alcohol ranges from about 10:90 to about 1:99.

30. The pharmaceutical composition of claim 26, wherein said disintegrant and said sugar alcohol and/or said saccharide are each present in the form of microparticles having an average particle size of about 30 μm or less.

31. The pharmaceutical composition of claim 26, wherein the ratio of said controlled-release particles to said rapidly disintegrating granules ranges from about 1:6 to about 1:2.

32. The pharmaceutical dosage form of claim 1, wherein the melperone is present as melperone hydrochloride, the alkaline buffer is disodium phosphate, and the water-insoluble polymer is ethylcellulose.

33. The pharmaceutical composition of claim 1, wherein the pharmaceutical composition when dissolution tested using United States Pharmacopoeia Apparatus 2 (paddles@50 rpm) in a 2-stage dissolution media (700 mL of 0.1N HCl for the first 2 hrs followed by testing in 900 mL buffer at pH 6.8 obtained by adding 200 mL of a pH modifier) at 37° C. exhibits a drug release profile substantially corresponding to the following pattern:
after an hour, no more than about 30% of the total active is released;
after 4 hrs, from about 40-70% of the total active is released;
after 8 hrs, from about 70-90% of the total active is released.

34. An orally disintegrating tablet comprising the composition of claim 1.

35. The pharmaceutical dosage form of claim 34, wherein said orally disintegrating tablet has a friability of less than about 1%.

36. The orally disintegrating tablet of claim 34, wherein said orally disintegrating tablet substantially disintegrates within about 60 seconds after contact with saliva in the oral cavity or with simulated saliva fluid.

37. The orally disintegrating tablet of claim 34, wherein said dosage form when dissolution tested using United States Pharmacopoeia Apparatus 2 (paddles@50 rpm) in a 2-stage dissolution media (700 mL of 0.1N HCl for the first 2 hrs followed by testing in 900 mL buffer at pH 6.8 obtained by adding 200 mL of a pH modifier) at 37° C. exhibits a drug release profile substantially corresponding to the following pattern:
after an hour, no more than about 30% of the total active is released;
after 4 hrs, from about 40-70% of the total active is released;
after 8 hrs, from about 70-90% of the total active is released.

38. A method of preparing the pharmaceutical composition of claim 1, comprising:
(a) preparing a core comprising melperone and/or a pharmaceutically acceptable salt, ester, or solvate thereof;
(b) coating the melperone core of step (a) with a layer comprising an alkaline buffer; and
(c) coating the alkaline-buffer layered core of step (b) with a controlled-release layer comprising a water-insoluble polymer.

39. The method of claim 38, wherein said step (a) comprises layering an inert bead with a solution comprising said weakly basic drug and optionally a polymeric binder.

40. The method of claim 38, wherein said step (c) further comprises a water-soluble polymer or an enteric polymer.

41. The method of claim 38, further comprising:
(d) mixing the microparticles with rapidly dispersing granules comprising a saccharide and/or sugar alcohol in combination with a disintegrant, thereby forming a compressible blend; and
(e) compressing said compressible blend into a tablet.

42. A method of treating psychosis and/or schizophrenia comprising administering to a patient in need thereof the composition of claim 1.
